(19) United States
(12) Patent Application Publication
(10) Pub. No.: US 2019/0027236 A1
(43) Pub. Date: Jan. 24, 2019

(54) PROVIDING A FAILURE PARAMETER IN MEDICAL CLOUD INFRASTRUCTURES

(71) Applicant: Siemens Healthcare GmbH, Erlangen (DE)

(72) Inventors: Vladyslav UKIS, Nuernberg (DE); Emilian ERTEL, Nuernberg (DE)

(73) Assignee: Siemens Healthcare GmbH, Erlangen (DE)

(21) Appl. No.: 16/033,320

(22) Filed: Jul. 12, 2018

(30) Foreign Application Priority Data
Jul. 21, 2017 (EP) 17182651.4

(51) Int. Cl. G16H 10/60; G06F 17/30; G06F 11/07; H04L 29/08

(52) U.S. Cl. CPC G16H 10/60; H04L 67/12; G06F 11/0766; G06F 17/3007

(57) ABSTRACT

A method is disclosed for providing a failure parameter. In an embodiment, the method includes receiving a medical data record by a first application module; determining a failure record by the first application module, upon the medical data record being non-processable by the first application module, the failure record being based on the data record; transmitting the failure record from the first application module to a failure analysis module; saving the failure record in a failure database by the failure analysis module; determining a failure parameter by the failure analysis module, the failure parameter being based on a statistical analysis of the failure database; and providing the failure parameter by the failure analysis module.

### PRIORITY STATEMENT

The present application hereby claims priority under 35 U.S.C. § 119 to European patent application number EP17182651.4 filed Jul. 21, 2017, the entire contents of which are hereby incorporated herein by reference.

### FIELD

At least one embodiment of the application generally relates to a method of providing a failure parameter in medical cloud infrastructures.

### BACKGROUND

In common cloud infrastructures, data records are transmitted between various modules as well processed by these modules in complicated workflows. In each of these transmitting and processing steps errors can occur, which make the data records non-processible for some of the modules of the cloud infrastructure. Examples for such errors are invalid or corrupt file formats, or the business logic of some application programs rejecting data records as invalid. As a result, some calculation results (in particular results of a statistical analysis) may differ from the expectation of the user, because data records that cannot be processed at any stage of the workflow in the cloud cannot be taken into account in the calculation.

This problem is particularly relevant within processing medical data. In the medical cloud platform teamplay, it is common to store anonymized examination results in the cloud and to perform a statistical analysis using this stored results, e.g. the average time of the medical examination, the average radiation dose absorbed by the patient due to the examination, or the number of radiation does outliers.

The data which is stored in the cloud originates from information systems located in the hospital, e.g. a PACS (acronym for “Picture Archiving and Communication System”), a HIS (acronym for “Hospital Information System”), a LIS (acronym for “Laboratory Information System”) or a RIS (acronym for “Radiology Information System”).

### SUMMARY

The inventors have recognized that if there are errors in the data processing of the cloud workflow, the basic set for the statistical analysis differs from the set of medical data records within the local systems. The inventors have recognized that this leads to incorrect data analysis results. Furthermore, the inventors have recognized that it is unclear for an inexperienced user of the system why there is a difference between the basic sets at all.

At least one embodiment of the present invention is directed to to taking the erroneous data records into account for the medical data analysis in order to improve the medical data analysis.

At least one embodiment of the present invention is directed to a method, a providing system, a computer program product and/or a computer-readable storage medium.

In the following, a solution according to at least one embodiment of the present invention is described with respect to the providing system as well as with respect to the method. Features, advantages or alternative embodiments herein can be assigned to claims for the providing systems can be improved with features described or claimed in the context of the method. In this case, the functional features of the method are embodied by objective units of the providing system.

In one embodiment, the invention relates to a method for providing a failure parameter, comprising:

receiving a medical data record by a first application module;

determining a failure record by the first application module, if the medical data record cannot be processed by the first application module, wherein the failure record is based on the data record;

transmitting the failure record from the first application module to a failure analysis module;

saving the failure record in a failure database by the failure analysis module;

determining a failure parameter by the failure analysis module, wherein the failure parameter is based on a statistical analysis of the failure database; and

providing the failure parameter by the failure analysis module.

In another embodiment, the invention relates to a providing system, comprising:

first application module, embodied for

receiving a medical data record,

determining a failure record, if the medical data record cannot be processed by the first application module, wherein the failure record is based on the data record, and

transmitting the failure record to a failure analysis module; and

failure analysis module, embodied for

saving the failure record in a failure database,

determining a failure parameter, wherein the failure parameter is based on a statistical analysis of the failure database, and

providing the failure parameter.

The invention relates in at least one embodiment to a computer program product comprising a computer program, the computer program being loadable into a storage unit of a providing system, including program code sections to make the providing system execute the method according to at least one embodiment of the invention when the computer program is executed in the providing system.

The invention relates in at least one embodiment to a computer-readable medium, on which program code sections of a computer program are saved, the program code sections being loadable into and/or executable in a providing system to make the providing system execute the method according to at least one embodiment of the invention when the program code sections are executed in providing system.

### BRIEF DESCRIPTION OF THE DRAWINGS

FIG. 1 shows a flow chart of an embodiment of the method for providing a failure parameter,

FIG. 2 shows a flow chart of an embodiment of the method for providing a data analysis,

FIG. 3 shows an embodiment of a providing system,

FIG. 4 shows a medical cloud data infrastructure without a providing system,

FIG. 5 shows the medical cloud data infrastructure with an embodiment of a providing system, and

FIG. 6 shows the medical cloud data infrastructure with another embodiment of a providing system.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without subdividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Bluray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In one embodiment, the invention relates to a method for providing a failure parameter, comprising:

receiving a medical data record by a first application module;

determining a failure record by the first application module, if the medical data record cannot be processed by the first application module, wherein the failure record is based on the data record;

transmitting the failure record from the first application module to a failure analysis module;

saving the failure record in a failure database by the failure analysis module;

determining a failure parameter by the failure analysis module, wherein the failure parameter is based on a statistical analysis of the failure database; and

providing the failure parameter by the failure analysis module.

In one example embodiment, the failure parameter is based on a set of failure parameters stored within the failure database. In one example embodiment, the failure record can be identical with the medical data record.

The inventors have recognized that by storing failure records within a failure database information about the number of errors and about reasons for the errors can be provided by the failure analysis module in terms of a failure parameter. This failure parameter can then be used for refining the analysis of the medical data.

In at least one embodiment, the step of transmitting the failure record from the first application module to the second application module comprises the steps of sending the failure record by the first application module and the step of receiving the failure record by the failure analysis module. In particular, the step of receiving the failure record is executed by an input interface of the failure analysis module. In particular, the step of saving the failure record is executed by a storage unit of the failure analysis module. In particular, the step of determining the failure parameter is executed by a calculation module of the failure analysis module. In particular, the step of providing the failure parameter is executed by an output interface of the failure analysis module.

In another possible embodiment of the invention, the failure record can be altered by the failure analysis module prior to storing in the failure database. The inventors have recognized that using a preprocessing of the failure record its data structure can be adapted to storage or computing time restrictions.

Another embodiment of the invention relates to a method for providing a data analysis, comprising all steps of the method for providing a failure parameter, furthermore comprising the steps of receiving the failure parameter by a second application module; and furthermore comprising the step of providing a medical data analysis based on the failure parameter by the second application module.

The first application module can be different from the second application module, but the first application module can also be identical with the second application module.

According to a further embodiment of the invention the failure record comprises a failure type, wherein the failure type indicates the reason for the medical data record being non-processible by the first application module. The inventors have recognized that by providing the failure type the information about the reason of the failure to process a medical data record can be stored and made accessible for further analysis.

According to a further embodiment of the invention, the medical data record comprises the result of a medical examination of a patient by a medical apparatus, wherein the failure record is an anonymized data record. In particular, the failure record comprises metadata of the data record, and the failure record does not comprise personal data of the patient. The inventors have recognized that by using only anonymized failure records the failure analysis module does not need to handle security and/or privacy restrictions, which makes it easier to implement and to use the failure analysis module.

According to a further embodiment of the invention, the medical data record comprises the result of a medical examination of a patient by a medical apparatus, wherein the failure record comprises metadata, and wherein the metadata relates to the medical apparatus, to a parameter of the medical examination and/or to the first application module. In particular the metadata does not comprise personal data or examination results. In particular, a parameter of the medical examination is an input value for the medical apparatus which influences the result of the medical examination. In particular, a parameter of the medical examination is not a result of the medical examination. In particular, the medical examination is a medical imaging examination. The inventors have recognized that by including metadata relating to the medical apparatus and/or to parameters of the medical examination, medical apparatuses and/or certain parameters of medical examinations that lead to erroneous data records can be identified quickly and easily.

According to a further embodiment of the invention, the metadata contains at least one of the following values of the medical examination:
start time and/or end time of the medical examination,
duration of the medical examination,
input parameter of the medical examination,
hardware components of the medical apparatus,
software version of the medical apparatus,
identifier of the first application module.

In particular, an input parameter of the medical examination can be an imaging protocol of the medical examination, if the medical examination is a medical imaging examination. The inventors have recognized that these values can be used for identifying medical examination procedures or medical apparatuses which lead to erroneous data records.

According to a further embodiment of the invention the medical data record cannot be processed by the first application module due to the medical data record exhibiting a non-readable format or due to the medical data record being classified as inconsistent by the first application module.

In particular, the medical data record exhibits a non-readable data format, if the medical data record is incomplete (e.g. to errors in the creation or the transmission of the medical data record), of if the first medical application is embodies to process only data records with a different data format and/or a different version of the data format. In other words, a medical data record with a non-readable data format cannot be processed by the first application module at all.

In particular, a medical data record is classified as inconsistent if at least one value of the medical data record (e.g. the duration of the medical examination or the radiation dose of the medical examination) lies outside a specified region or interval of this value. Such regions or intervals are usually specified in the first application module to prevent medical data records not relating to a medical examination to be processed. In other words, a medical data record is classified as inconsistent, if the medical data record can theoretically be processed by the first application module, but is rejected according to some program logic of the first application module. The inventors have recognized that these two reasons for the first application module not being able to process the medical data record cover a significant majority of all possible reasons, so restricting to these two reasons leads to a simpler but yet precise method for error management.

According to a further embodiment of the invention, the failure parameter relates to the number of failure records in the failure database. The inventors have recognized that this number can be calculated very fast and efficiently, and gives valuable insight into the total defectiveness of the system at the same time.

According to a further embodiment of the invention, the failure record comprises a module identifier of the first application module, wherein the failure parameter comprises the number of failure records in the failure database comprising a given module identifier. In particular, the failure parameter can comprise a list of module identifiers and the number of failure records comprising the respective module identifier. The inventors have recognized that by including the module identifier into the failure record and by resting the failure parameter on the module identifier an erroneous first application module can be identified quickly and easily.

According to a further embodiment of the invention, the medical data record comprises the results of a medical examination by an medical apparatus, wherein the failure record comprises an apparatus identifier of the medical apparatus, and wherein the failure parameter comprises the number of failure records in the failure database comprising a given apparatus identifier. In particular, the failure parameter can comprise a list of apparatus identifiers and the number of failure records comprising the respective apparatus identifier. The inventors have recognized that by including the apparatus identifier into the failure record and by resting the failure parameter on the module identifier an erroneous medical apparatus can be identified quickly and easily.

According to a further possible embodiment of the invention, the medical data record comprises the results of a medical examination by a medical apparatus, wherein the failure record comprises a module identifier and an apparatus identifier of the medical apparatus, and wherein the failure parameter comprises the number of failure records comprising a given module identifier and a given apparatus identifier. In particular, the failure parameter can comprise a list of module identifiers, apparatus identifiers and the number of failure records comprising the respective module identifier and the respective apparatus identifier.

In another embodiment, the invention relates to a providing system, comprising:

first application module, embodied for

receiving a medical data record,

determining a failure record, if the medical data record cannot be processed by the first application module, wherein the failure record is based on the data record, and

transmitting the failure record to a failure analysis module; and

failure analysis module, embodied for

saving the failure record in a failure database,

determining a failure parameter, wherein the failure parameter is based on a statistical analysis of the failure database, and

providing the failure parameter.

According to a further embodiment of the invention the providing system comprises:
second application module, embodied for receiving (REC-2) the failure parameter, and furthermore embodied for providing (PROV-2) a medical data analysis based on the failure parameter.

In at least one embodiment, the providing system can be embodied to execute the method according to at least one embodiment of the invention and its aspects. The providing system is embodied to execute the method its aspects by the first application module, the failure analysis module and the optional second application module being embodied to execute the respective method steps.

The providing system can be realized as a data processing system or as a part of a data processing system. Such a data processing system can, for example, comprise a cloud-computing system, a computer network, a computer, a tablet computer, a smartphone or the like. The providing system can comprise hardware and/or software. The hardware can be, for example, a processor system, a memory system and combinations thereof. The hardware can be configurable by the software and/or be operable by the software.

A possible further embodiment of the invention relates to the providing system, wherein the failure analysis module comprises a first failure analysis sub-module and a second failure analysis sub-module, wherein the failure database comprises a first failure sub-database and a second failure sub-database, wherein the first failure analysis sub-module and the first failure sub-database are located in a hospital environment, and wherein the second failure analysis module and the second failure sub-database are located in a public cloud environment. In particular, both the first failure analysis sub-module and the first failure sub-database are embodied as the failure analysis module and the failure database according to one of the embodiments of the invention. In particular, both the second failure analysis sub-module and the second failure sub-database are embodied as the failure analysis module and the failure database according to one of the embodiments of the invention. The inventors have recognized that by using a separate module within the private hospital environment and within the public cloud environment different failure records can be saved in different environments, so that data privacy regulations can be fulfilled.

The invention relates in at least one embodiment to a computer program product comprising a computer program, the computer program being loadable into a storage unit of a providing system, including program code sections to make the providing system execute the method according to at least one embodiment of the invention when the computer program is executed in the providing system.

The invention relates in at least one embodiment to a computer-readable medium, on which program code sections of a computer program are saved, the program code sections being loadable into and/or executable in a providing system to make the providing system execute the method according to at least one embodiment of the invention when the program code sections are executed in providing system.

The realization of at least one embodiment of the invention by a computer program product and/or a computer-readable medium has the advantage that already existing providing systems can be easily adopted by software updates in order to work as proposed by at least one embodiment of the invention.

The computer program product can be, for example, a computer program or comprise another element apart from the computer program. This other element can be hardware, for example a memory device, on which the computer program is stored, a hardware key for using the computer program and the like, and/or software, for example a documentation or a software key for using the computer program.

At least one embodiment of the invention relates, in another possible embodiment, to a first application module, embodied for receiving a medical data record, furthermore embodied for determining a failure record, if the medical data record cannot be processed by the first application module, wherein the failure record is based on the data record, furthermore embodied for transmitting the failure record to a failure analysis module.

At least one embodiment of the invention relates, in another possible embodiment, to a failure analysis module, embodied for saving the failure record received from the first application module in a failure database, furthermore embodied for determining a failure parameter, wherein the failure parameter is based on a statistical analysis of the failure database, furthermore embodied for providing the failure parameter, in particular embodied for providing the failure parameter to a second application module.

At least one embodiment of the invention relates, in another possible embodiment, to a second application module, embodied for receiving the failure parameter, in particular embodied for receiving the failure parameter from a failure analysis module, furthermore embodied for providing a medical data analysis based on the failure parameter.

A medical data record can comprise data relating to a medical examination, in particular personal data of the patient examined (e.g. the patient name, the patient age, the patient sex), medical examination results (e.g. images or laboratory values), and metadata of the examination. In particular, the medical data record can be formatted according to a medical data format, e.g. DICOM (acronym for “Digital Imaging and Communications in Medicine”) or HL7 (acronym for “Health Level Seven”).

Metadata of a medical examination or a respective medical data record can comprise all data which is not related to the patient of the medical examination and which is not related to the result of the medical examination. For example, the metadata of a medical examination can comprise an identifier of the medical apparatus used for the medical examination, the procedure used for the medical examination (e.g. the imaging protocol, or the workflow within the laboratory for a sample), the start time, the end time and/or the duration of the medical examination, a radiologic dose administered by the medical examination. In particular, metadata relates to the medical apparatus, a parameter of the medical examination and/or the first application module.

A failure record is usually based on a medical data record, and relates to an error occurred during the processing of the medical data record. A failure record can comprise the medical data record in whole or in part. Furthermore, a failure record can also comprise additional data or parameters; in particular data related with the error occurred during the processing of the medical data record.

The private hospital environment (or short, the hospital environment, or the hospital IT infrastructure) is particularly the IT infrastructure located directly at a hospital, which can be accessed only by local users and by means of remote maintenance. The private hospital environment can comprise medical modalities, servers (e.g. for data storage and for computations), clients and the network connecting different entities, as well as software programs running on these hardware components. The private hospital environment can be identified with an intranet. The private hospital environment can comprise components which are embodied to receive data from outside the private hospital environment or to send data to the outside of the private hospital environment; usually these components are embodied to prevent unauthorized access to the private hospital environment.

The public cloud environment (or short the cloud environment, or the cloud IT infrastructure) is particularly the IT infrastructure located in the cloud, embodied to communicate and interact with one or several private hospital environments. The public cloud infrastructure can comprise servers (e.g. for data storage and for computations), clients and the network connecting different entities, as well as software programs running on these hardware components. The private hospital environment can be a part of the internet. Usually the public cloud environment is embodied to allow access only to authorized users.

A cloud or a cloud environment comprises computing services and/or storage services made available by a service provider, which can be used by one or several service users according to their demand. In order to make these services available, the service provider usually operates physical computing units (e.g. comprising microprocessors) and storage units (e.g. comprising hard disks), which can then be used by the service providers by predefined interfaces. Such interfaces can relate on the usage of virtual machines or API calls. Known variants of a cloud environment comprise IaaS (acronym for “Infrastructure as a Service”), PaaS (acronym for “Platform as a Service”) or SaaS (acronym for “Software as a Service”).

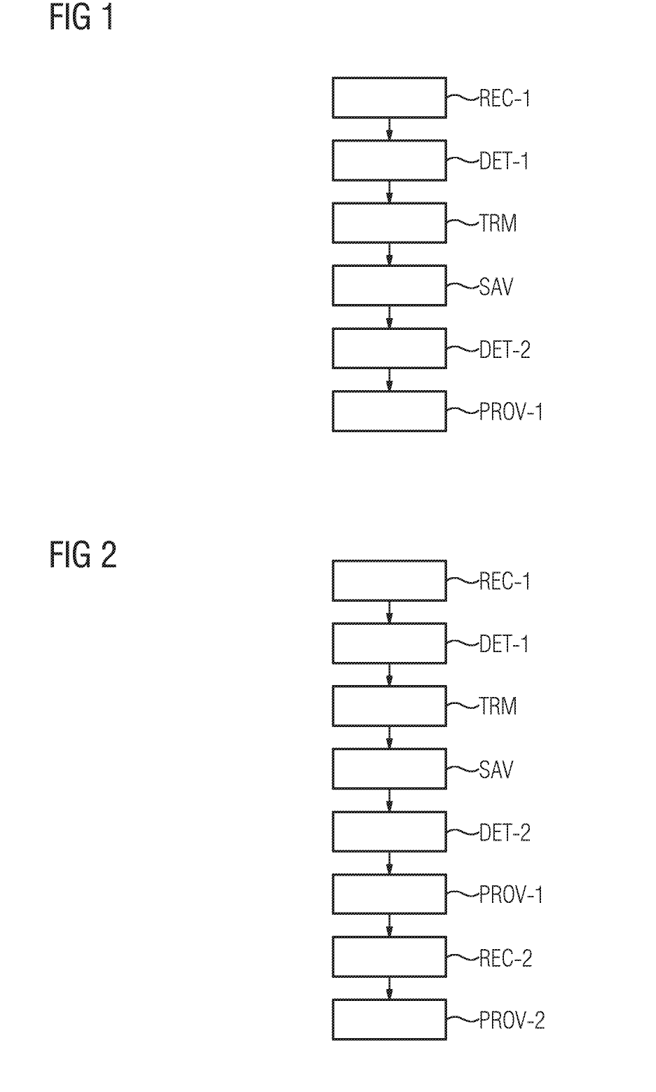

FIG. 1 shows a flow chart of an embodiment of the method for providing a failure parameter. The first step of the displayed embodiment is receiving REC-1 a medical data record by a first application module 320. In this embodiment, the first application module 320 is an instance of a software application, which provides a software interface for receiving medical data records. In this embodiment, a medical data record is the result of a medical imaging examination with a medical imaging apparatus, here a computed tomography scanner. Alternatively, the medical imaging apparatus can be a magnetic resonance scanner, an X-ray scanner or an ultrasound scanner. The medical data record can also be the result of another medical examination, e.g. laboratory results or results from medical apparatuses for point-of-care diagnostics (e.g. measuring blood glucose, temperature, oxygen saturation of the blood, heartbeat sequence, etc.). The medical data record can also be a patient data record e.g. from a hospital information system, comprising personal data and results from previous examinations.

The second step of the displayed embodiment is determining DET-1 a failure record 341.1, 341.2 by the first application module 320, if the medical data record cannot be processed by the first application module 320. In this embodiment the failure record 341.1, 341.2 comprises an identifier 342.1, 342.2 of the first application module 320, an identifier 343.1, 343.2 of the medical apparatus, and a failure type 344.1, 344.2 that indicates the reason the medical data record cannot be processed by the first application module 320.

The identifier 342.1, 342.2 of the first application module 320 comprises in this embodiment the name of the software program and the version of the software program in a string, for example “ABC-Software v1.1”, where “ABC-Software” is the name of the software program and “v1.1” is the version of the software program. The identifier 343.1, 343.2 of the medical apparatus comprises a serial number of the medical apparatus. In particular, the identifier 343.1, 343.2 of the medical apparatus can also comprise the hardware components used in the medical apparatus and/or the version of the operating software of the medical apparatus.

In this embodiment the failure type 344.1, 344.2 is a string comprising an error message. In this embodiment two reasons for non-processability are treated, the first one is that the medical data record is corrupt or incomplete (error message “DATA_CORRUPT”), the second one is that the medical data record does not comply with the rules for a valid medical data record (error message “DATA_INVALID”). In this embodiment, a medical data record does not comply with the rules if certain parameters of the medical data record lie outside predefined boundaries, or if certain conditions are met or not met. E.g. some post-processing algorithms overwrite the start and the end time of the examination within a DICOM-file with times from the post-processing, in this case it is not possible to evaluate this data for obtaining usage statistics about medical apparatuses. Alternatively it is possible to use more granular error messages which describe the reason for non-processability in more detail (e.g. the error message “DATA_INVALID_EXAMINATION_TIME” for an examination time outside of the valid interval for an examination time, or “DATA_INVALID_ANONYMIYATION” for a non-anonymized medical data record).

The third step of this embodiment is transmitting TRM the failure record 341.1, 341.2 from the first application module 320 to a failure analysis module 330. In particular, the failure record 341.1, 341.2 is transmitted to an input interface 331 of the failure analysis module 330. Since the failure record 341.1, 341.2 in this embodiment is a complex data type comprising several data fields (e.g. the identifier 342.1, 342.2 of the first application module 320, for the identifier 343.1, 343.2 of the medical apparatus, and for the failure type 344.1, 344.2), the failure record 341.1, 341.2 has to be serialized before transmitting TRM it from the first application module 320 to the failure analysis module 330, or alternatively stored in a common data format (e.g. XML).

The fourth step of this embodiment is saving SAV the failure record 341.1, 341.2 in a failure database 340 by the failure analysis module 330. The failure records 341.1, 341.2 are stored in a relational database (e.g. using SQL commands), but they can also be stored in a non-relational database or as single files on a hard drive.

The fifth step of this embodiment is determining DET-2 a failure parameter by the failure analysis module 330, wherein the failure parameter is based on a statistical analysis of the failure database 340. In this embodiment a statistical analysis is based on counting the number of failure records 341.1, 341.2 with a certain property. One can also use more complicated statistical analysis methods, e.g. calculating means, variances or frequencies. In this embodiment, the statistical analysis results in a list of module identifiers 342.1, 342.2, apparatus identifiers 343.1, 343.2 and the number of failure records 341.1, 341.2 in the failure database 340 comprising the respective module identifier 342.1, 342.2 and the respective apparatus identifier 343.1, 343.2.

Suppose that there are two first application modules 320 with module identifier “module_1” and “module_2”, and two medical apparatuses with apparatus identifier “apparatus_1” and “apparatus_2”. The resulting list is then

(“module_1”, “apparatus_1”): “number_1_1”
(“module_1”, “apparatus_2”): “number_1_2”
(“module_2”, “apparatus_1”): “number_2_1”
(“module_2”, “apparatus_2”): “number_2_2”

where “number_i_j” is the number of failure records 341.1, 341.2 in the failure database 340 comprising the module identifier “module_i” and the apparatus identifier “apparatus_j”. So if the failure database 340 has the following entries

(“module_1”, “apparatus_1”, “DATA_CORRUPT”)
(“module_1”, “apparatus_1”, “DATA_CORRUPT”)
(“module_2”, “apparatus_1”, “DATA_INVALID”)
(“module_2”, “apparatus_2”, “DATA_INVALID”)

then the resulting list of failure parameters is:

(“module_1”, “apparatus_1”): 2
(“module_1”, “apparatus_2”): 0
(“module_2”, “apparatus_1”): 1
(“module_2”, “apparatus_2”): 1

Optionally, the failure records 341.1, 341.2 can comprise other metadata of the medical examination, e.g. the duration of the medical examination, the starting time of the medical examination, the type of the examination protocol used for the medical examination, or the name of the medical professional executing the medical examination. In this case, the failure parameter can comprise this additional metadata, or also analysis with respect to this metadata. For example there might be a correlation between the examination protocol used and the invalidity of the medical data record, which can be detected using statistical analysis of the failure parameter.

The last step of this embodiment is providing PROV-1 the failure parameter by the failure analysis module 330. In particular, the failure parameter can be provided by an output interface 332 of the failure analysis module 330. In this embodiment, the providing PROV-1 comprises sending the failure parameter to a second application module 350, wherein the sending is executed in answer to an API (acronym for “Application Programming Interface”) call by the second application module 350. Alternatively the failure parameter can also be stored in a storage which is externally available, e.g. on a web-server (which can be accessed e.g. by a web-service or a HTTP-request).

FIG. 2 shows a flow chart of an embodiment of the method for providing a data analysis. The steps of receiving REC-1, determining DET-1, transmitting TRM, saving SAV, determining DET-2 and providing PROV-1 are the same as in the embodiment of the method for providing a failure parameter displayed and explained in FIG. 1.

The seventh step of the embodiment of the method for providing a data analysis is receiving REC-2 the failure parameter by the second application module 350. In this embodiment, the first application module 320 and the second application module 350 are not identically; alternatively the first application module 320 and the second application module 350 can be identically. Furthermore, here the step of receiving REC-2 is executed as an API call to the failure analysis module 330 and the subsequent receiving of the answer to the API call.

The last step of the embodiment of the method for providing a data analysis is providing PROV-2 a medical data analysis based on the failure parameter by the second application module 350. In this embodiment, the second application module 350 provides a data analysis about the usage of medical apparatuses, which comprises for each considered medical apparatus a percentage of time in which the respective medical apparatus executes medical examinations. For example, if there are 20 medical examinations with a medical apparatus within a certain day, wherein only 18 examinations can be processed properly by the first application module 320 (which in this embodiment anonymizes the medical data records and stores the anonymized medical data records in the cloud), then the corresponding medical data analysis is: “77% usage (based on 18 analyzed of 20 total data records)”. Here the addressee of the medical data analysis is informed that the usage statistics is not calculated based on the total number of examinations on this day, but only on the fraction of valid examinations.

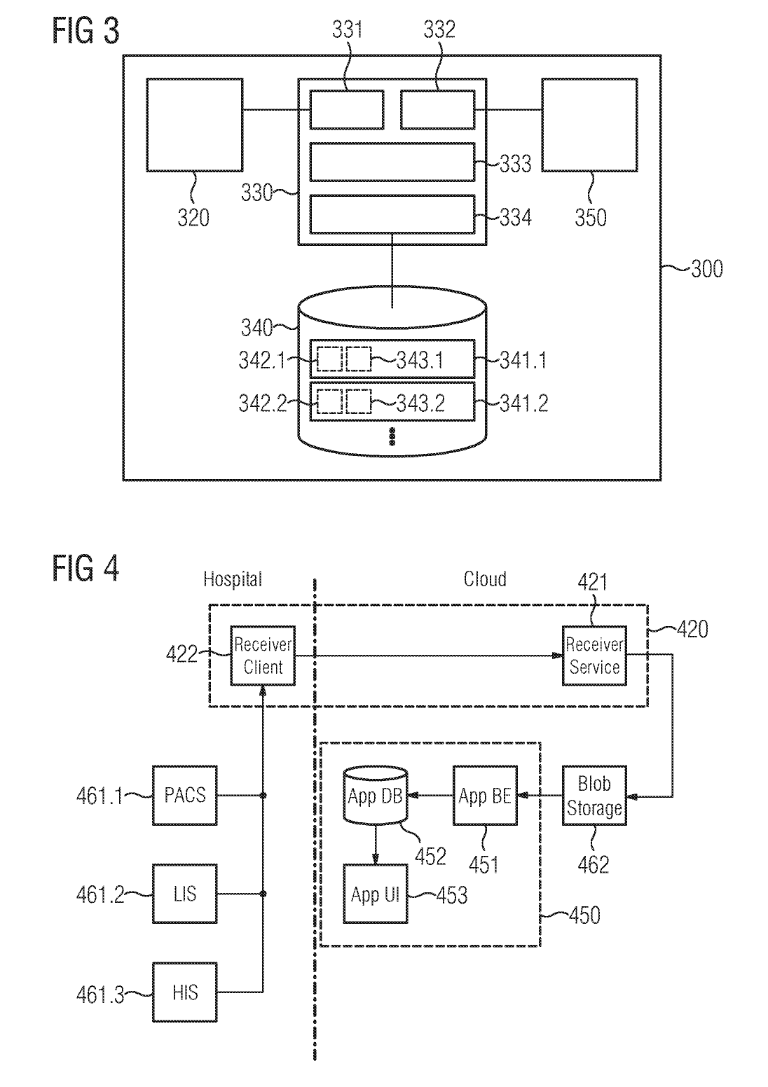

FIG. 3 shows a providing system 300 comprising a first application module 320, a failure analysis module 330 a failure database 340 and a second application module 350.

In this embodiment the first application module 320, the second application module 350 and the failure analysis module 330 are software applications within a cloud environment. Alternatively, the providing system 300, the first application module 320, the second application module 350 and/or the failure analysis module 330 can be hardware entities, e.g. (personal) computers, workstations, virtual machines running on a host hardware, microcontrollers, or integrated circuits. As an alternative, the providing system 300 and/or the failure analysis module 330 can be a real or a virtual group of computers (the technical term for a real group of computers is “cluster”, the technical term for a virtual group of computers is “cloud”).

The communication between the first application module 320, the second application module 350 and the failure analysis module 330 is in this embodiment executed via API (“Application Programming Interface”) calls, alternatively the communication can be done via web-services or by providing data records in public available storages (accessable e.g. by “File Transfer Protocoll” FTP or “Hypertext Transfer Protocoll” HTTP).

In this embodiment, the failure analysis module 330 comprises an input interface 331, an output interface 332, a calculation module 333 and a storage module 334. In this embodiment, the input interface 331, the output interface 332, the calculation module 333 and the storage module 334 are embodied as software sub-modules of the failure analysis module 330, they can also be embodied as hardware sub-modules if the failure analysis module 330 is a hardware module.

An input interface 331 and an output interface 332 can be embodies as a hardware interface or as a software interface (e.g. PCI-Bus, USB or Firewire). In general, a calculation module 333 can comprise hardware elements and software elements, for example a microprocessor or a field programmable gate array. A hardware memory module 334 can be embodied as non-permanent main memory (e.g. random access memory) or as permanent mass storage (e.g. hard disk, USB stick, SD card, solid state disk).

In the displayed embodiment, the failure database 340 is embodied separately from the failure analysis module 330, e.g. as a database server or as a database service. For databases several standards are known, e.g. relational databases servers or services using e.g. SQL (“Structured Query Language”), or NoSQL database servers or services storing data e.g. in key-value-pairs or using graph models, only partially guaranteeing the consistency and the availability of the stored data records. Alternatively the failure database 340 can be embodied within the storage module 334 of the failure analysis module 330.

In this embodiment, the failure database 340 contains failure records 341.1, 341.2, each failure record 341.1, 341.2 comprising a module identifier 342.1, 342.2 and an apparatus identifier 343.1, 343.2 as metadata of the respective medical data record. If the failure database 340 is a relational database, it is favorable to use primary keys as module identifiers 342.1, 342.2 and apparatus identifiers 343.1, 343.2, and to store details of the modules and apparatuses in separate tables (to ensure a proper normalization of the database) with respect to the respective primary key.

FIG. 4 shows a graphical sketch of the functionality of a cloud framework for storing and processing medical data records comprising a hospital environment and a cloud environment without a failure analysis module. On the left side of FIG. 4 software and/or hardware modules are displayed which are parts of a local hospital environment; on the right hand side of FIG. 4 software and/or hardware modules are displayed with are parts of the cloud environment. Due to legal data privacy restrictions personal data may only be stored in the hospital environment, and not in a cloud environment.

The hospital environment comprises data sources 461.1, 461.2, 461.3, comprising a PACS 461.1 (“Picture Archiving and Communication System”), a LIS 461.2 (“Laboratory Information System”) and a HIS 461.3 (“Hospital information System”). Furthermore, the hospital environment comprises a receiver client 422, which gathers data records from the data sources 461.1, 461.2, 461.3, anonymizes them and sends them to the cloud environment. The receiver client 422 can be local software installed on one or more of the data sources 461.1, 461.2, 461.3, in this embodiment it is embodied as a separate server located in the internal network of a hospital, so that it can access the data sources 461.1, 461.2, 461.3.

The cloud environment comprises a receiver service 421 which receives the anonymized data records from the receiver client 422. In this embodiment, the receiver service 421, the receiver client 422 and their communication constitute the first application module 420. Furthermore, the cloud environment comprises a BLOB (acronym for “Binary Large Object”) storage 462 which is embodied to store the medical data records received by the receiver service 421.

Furthermore, the cloud environment comprises a second application module 450, comprising an application backend 451, an application database 452 and an application frontend 453. The application backend 451 is embodied to access the BLOB storage 462 in order to provide a statistical analysis of the medical data records contained in the BLOB storage 462. The results of the statistical analysis are then stored in the application database 452, which can be accessed by the application frontend 453 in order to display the results of the statistical analysis.

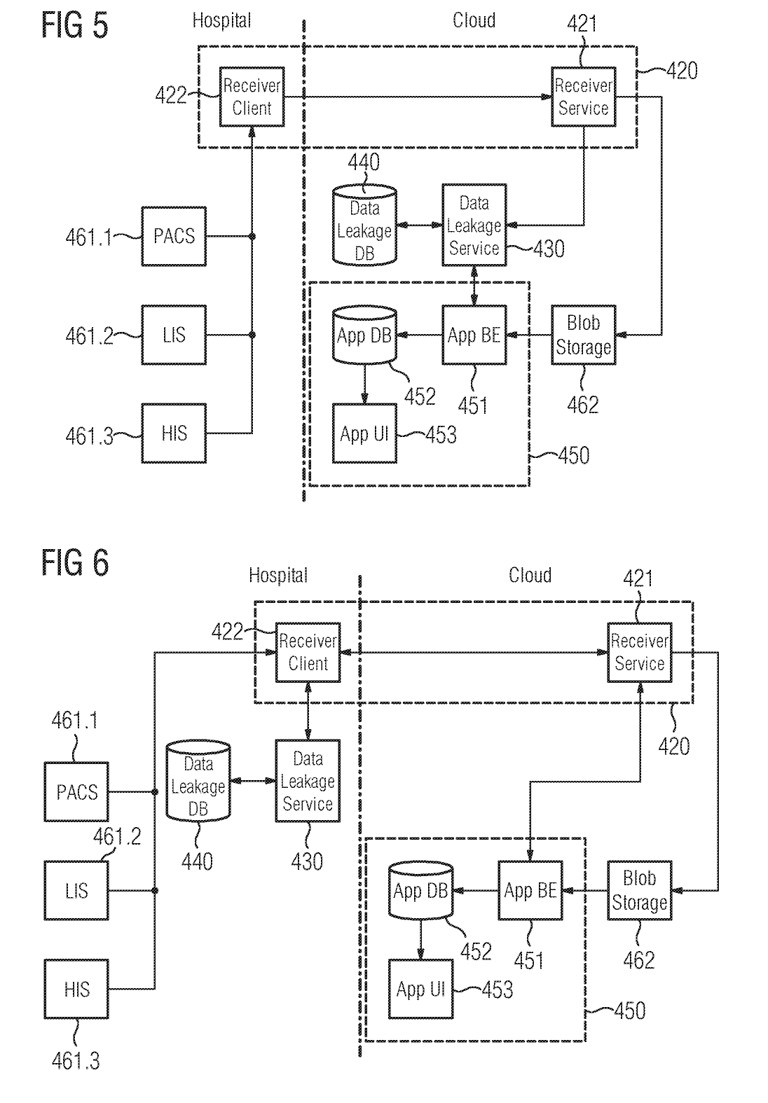

FIG. 5 shows a graphical sketch of the functionality of a cloud framework for storing and processing medical data records comprising a hospital environment and a cloud environment, wherein the cloud environment comprises a data leakage service 430 as a failure analysis module and a data leakage database 440 as a failure database. This cloud framework is an extension of the cloud framework depicted in FIG. 4.

In this embodiment, the data leakage service 430 is in communication with the receiver service 421 and with the application backend 451. The first application module 420, comprising the receiver service 421 and the receiver client 422, processes data records from the hospital data sources 461.1, 461.2, 461.3. Therein the receiver client 422 anonymizes the data records and sends the anonymized data records to the receiver service 421. The receiver service 421 checks the anonymized data records for compliance with predefined requirements, in this case that the duration of the medical examination lies in predefined boundaries. If the duration of the medical examination is within the predefined boundaries (and all other predefined checks are positive), the medical data record will be sent to the BLOB storage 462. If the duration of the medical examination is not within the predefined boundaries, the data record is considered as invalid, and a failure record 341.1, 341.2 comprising the data record is sent to the data leakage service 430 and stored in the data leakage database 440 by the data leakage service 430.

In this embodiment, the application backend 451 is embodied to provide a statistical analysis about the usage of one or several medical apparatuses, e.g. the relation of the total examination time to the operation time of the medical apparatus. Therefor the application backend 451 can communicate with the BLOB storage 462 and with the data leakage service 430. From the medical data records stored in the BLOB storage 462 the relation of the total examination time to the total operation time can be calculated, and by querying the data leakage service 430 with the medical apparatus, the number of failure records 341.1, 341.2 comprising the identifier of the respective medical apparatus can be obtained by the application backend 451. So the application frontend 453 can display the ratio of total examination time to the total operation time as well as the number of failure records 341.1, 341.2, where the number of failure records 341.1, 341.2 can be used for estimating the influence of erroneous medical data records on the statistical analysis.

In this embodiment, also the receiver client 422 can send failure records 341.1, 341.2 to the data leakage service 430 through the receiver service 421, e.g. if the anonymization of the medical data record is not successful. In this case it is advantageous to include only metadata of the medical data record to the data leakage service 430, wherein the metadata does not contain personal data of a patient, so that the failure records 341.1, 341.2 can be stored in the cloud environment without violating data privacy regulations.

FIG. 6 shows a graphical sketch of the functionality of a cloud framework for storing and processing medical data records comprising a hospital environment and a cloud environment, wherein the hospital environment comprises a data leakage service 430 as a failure analysis module and a data leakage database 440 as a failure database. This cloud framework is an extension of the cloud framework depicted in FIG. 4.

In this embodiment, the data leakage service 430 is only in communication with the receiver client 422. The application backend 451 can access the data leakage service 430 through the receiver service 421 and the receiver client 422. If the receiver client 422 cannot process a medical data record from one of the data sources 461.1, 461.2, 461.3 properly, because e.g. the anonymization procedure fails, it sends a failure record 341.1, 341.2 to the data leakage service 430, which stores the failure record 341.1, 341.2 in the data leakage database 440. By keeping the data leakage service 430 and the data leakage database 440 within the hospital environment, it is possible to store non-anonymized medical data records in the data leakage database 440 without violating data privacy constraints.

The data leakage service 430 can provide the failure parameter to the application backend 451 through the receiver client 422 and the receiver service 421. In this embodiment, the data leakage service 430 ensures that the failure parameter does not contain any personal data, alternatively the receiver client 422 can anonymize the failure parameter. If the failure parameter comprises e.g. the total number of failure records 341.1, 341.2, the application backend 451 or the application frontend 453 can use this total number to allow for estimating a user the impact of the erroneous data records on the statistical analysis.

By operating the data leakage service 430 and the data leakage database 440 within the hospital environment, it is possible that the failure record 341.1, 341.2 comprises the complete medical data record including personal data, without storing the personal data in the cloud. Using the complete medical data records is useful for locating the cause for the non-processability of the medical data record. For the error search, on the one hand, the failure records 341.1, 341.2 can be accessed directly from within the hospital environment; on the other hand, the failure records 341.1, 341.2 can also be accessed from within the cloud environment through the receiver service 421 and the receiver client 422, if certain authentication conditions are met (e.g. the user is authenticated as a technical administrator) and checked by the local receiver client 422 within the hospital environment.

In particular, the arrows in FIG. 4, FIG. 5 and FIG. 6 indicate the flow of data between the single components within the cloud framework.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for providing a failure parameter, comprising:
receiving a medical data record by a first application module;
determining a failure record by the first application module, upon the medical data record being non-processible by the first application module, the failure record being based on the medical data record;
transmitting the failure record from the first application module to a failure analysis module;
saving the failure record in a failure database by the failure analysis module;
determining a failure parameter by the failure analysis module, the failure parameter being based on a statistical analysis of the failure database; and
providing the failure parameter by the failure analysis module.

2. The method of claim 1, further comprising:
receiving the failure parameter by a second application module; and
providing a medical data analysis based on the failure parameter by the second application module.

3. The method of claim 1, wherein the failure record includes a failure type, and wherein the failure type indicates a reason for the medical data record being non-processible by the first application module.

4. The method of claim 1, wherein the medical data record comprises a result of a medical examination of a patient by a medical apparatus, and wherein the failure record is an anonymized data record.

5. The method of claim 1, wherein the medical data record includes a result of a medical examination of a patient by a medical apparatus, wherein the failure record includes metadata, and wherein the metadata relates to at least one of the medical apparatus, a parameter of the medical examination and the first application module.

6. The method of claim 5, wherein the metadata contains at least one of the following values of the medical examination:
at least one of start time and end time of the medical examination,
duration of the medical examination,
input parameter of the medical examination,
hardware components of the medical apparatus,
software version of the medical apparatus, and
identifier of the first application module.

7. The method of claim 1, wherein the medical data record is non-processible by the first application module upon the medical data record exhibiting a non-readable format or upon the medical data record being classified as inconsistent by the first application module.

8. The method of claim 1, wherein the failure parameter relates to a number of failure records in the failure database.

9. The method of claim 8, wherein the failure record includes a module identifier of the first application module, and wherein the failure parameter includes the number of failure records in the failure database including a module identifier.

10. The method of claim 8, wherein the medical data record includes results of an imaging medical examination by an medical apparatus, wherein the failure record includes an apparatus identifier of the medical apparatus, and wherein the failure parameter includes the number of failure records in the failure database includes a given apparatus identifier.

11. A providing system, comprising:
first application module, embodied to
receive a medical data record,
determine a failure record upon the medical data record being non-processible by the first application module, the failure record being based on the medical data record, and
transmit the failure record to a failure analysis module; and
failure analysis module, embodied for
save the failure record in a failure database,
determine a failure parameter, the failure parameter being based on a statistical analysis of the failure database,
provide the failure parameter.

12. The providing system of claim 11, comprising:
second application module, to receive the failure parameter, and to provide a medical data analysis based on the failure parameter.

13. The providing system of claim 11, wherein the failure record includes a failure type, and wherein the failure type indicates a reason for the medical data record being non-processible by the first application module.

14. A non-transitory computer program product storing a computer program, the computer program being loadable into a storage unit of a providing system, including program code sections to configure the providing system to execute the method of claim 1 when the computer program is executed in the providing system.

15. A non-transitory computer-readable medium, storing program code sections of a computer program, the program code sections being at least one of loadable into and executable in a providing system to configure the providing system to execute the method of claim 1 when the program code sections are executed in the providing system.

16. The method of claim 2, wherein the failure record includes a failure type, and wherein the failure type indicates a reason for the medical data record being non-processible by the first application module.

17. The method of claim 2, wherein the medical data record comprises a result of a medical examination of a patient by a medical apparatus, and wherein the failure record is an anonymized data record.

18. The method of claim 2, wherein the medical data record includes a result of a medical examination of a patient by a medical apparatus, wherein the failure record includes metadata, and wherein the metadata relates to at least one of the medical apparatus, a parameter of the medical examination and the first application module.

19. The method of claim 2, wherein the medical data record is non-processible by the first application module upon the medical data record exhibiting a non-readable format or upon the medical data record being classified as inconsistent by the first application module.

20. The method of claim 2, wherein the failure parameter relates to a number of failure records in the failure database.
